(19) United States
(12) Patent Application Publication
(10) Pub. No.: US 2008/0045594 A1
(43) Pub. Date: Feb. 21, 2008

(54) Use of at Least One Conjugated Triene-Containing Fatty Acid for Preparing a Medicine for Treating Inflammation

(75) Inventors: Antoine Piccirilli, Villennes/Seine (FR); Nathalie Piccardi, Arceau (FR); Philippe Miska, Versailles (FR)

Correspondence Address: FOLEY AND LARDNER LLP;SUITE 500, 3000 K STREET NW, WASHINGTON, DC, 20007, US

(73) Assignee: LABORATOIRES EXPANSCIENCE, Courbevoie (FR)

(21) Appl. No.: 11/572,769

(22) PCT Filed: Jul. 20, 2005
(86) PCT No.: PCT/FR05/01859

§ 371 Date: Jan. 26, 2007

(30) Foreign Application Priority Data
Jul. 26, 2004 (FR) 0408230

(51) Int. Cl. A61K 31/20; A61P 29/00; C07C 51/41

(52) U.S. Cl. 514/558; 554/12

(57) ABSTRACT

The invention concerns the use of at least one conjugated triene-containing fatty acid for preparing a medicine for treating inflammation selected among the group consisting of alpha-eleostearic acid, catalpic acid, calendic acid, jacaric acid, licanic acid and beta-eleostearic acid for making a medicine for treating inflamatory diseases and/or metabolic disorders following an inflammation. The inflammatory diseases and/or metabolic disorders following an inflammation may be skin cancers, sun burn, benign summer light eruption, allergic and/or irritative reactions, gingivitis and periodontitis, vulvitis and vaginitis or arthritis and arthrosis. The medicine also enables healing to be promoted. Said fatty acid conjugates can also be used in the cosmetic treatment of cellulitis.

The object of the invention is the use of at least one conjugated triene-containing and optionally oxygenated fatty acid of synthetic and/or natural origin, for making a drug intended to treat inflammatory diseases and/or metabolic disorders consecutive to inflammation.

The most common triene-containing fatty acid which is found in plants is α-linolenic acid. Several conjugated isomers of α-linolenic acid may be found in certain plant formulations and they sometimes become majority constituents of these vegetable oils. Catalpic acids from catalpa ovata, punicic acid from Punica granatum, jacaric acid from Jacaranda ninosisonia and calendic acid from Calendula officinalis, pomegranate (Punica granutum L.), catalpa (Catalpa ovata G.), balsam, . . . may notably be mentioned.

Conjugated triene-containing fatty acids, such as α-linolenic acid or calendic acid, have already been used in pharmaceutical compositions intended for treating and/or preventing cholesterolemia.

The inflammatory process is the whole of the reaction phenomena triggered in a pluricellular living organism by aggression of any pathogenic agent. This is an omni-tissue phenomenon which preferentially occurs in conjunctive tissue which normally tends to limit and repair the effect of the aggression. It terminates with repair or healing of the lesion.

The inflammation may be caused by physical aggressions (such as heat, cold, ionizing radiations), chemical aggressions (caused by acid or basic compounds, bacterial toxins). It may be the consequence of an infection related to the presence in the organism, of pathogenic living organisms such as bacteria, viruses, parasites or fungi. It may be caused by a secondary immune reaction to re-introduction of an antigen into the organism. Finally, it may be the consequence of tissue necrosis, itself secondary to many causes, for example an arterial occlusion.

The causes are multiple and represent pathogenic agents. They determine cell or tissue lesions which will trigger the inflammation:
physical causes (trauma, heat, cold, radiation, electric current);
trophic causes through lack of vascularization;
chemical causes (acids, bases, exogenic or endogenic “foreign” bodies);
biological causes (germs, bacteria, viruses, parasites, fungi);
immune conflict.

In the inflammation, the pathogenic agent may be endogenic or exogenic, and the infectious causes (microorganisms) are only a small portion of the causes of inflammation. Certain causes determine lesions, the morphology of which is peculiar, hence the notion of specific inflammation. On the other hand, a same pathogenic agent may cause different inflammatory reactions depending on the host, hence the importance of the factors related to the host (promoting factors or protective factors).

The progress of the inflammatory process develops in three successive stages:
a stage characterized by vascular/haematic reactions;
a stage characterized by cell reactions (productive phase);
a healing or regeneration stage.

When ultraviolet radiation reaches the skin, a portion of it is reflected from the surface. The remainder of the radiation is diffused into the tissues just below the surface of the skin. A fraction of this radiation is absorbed by the living cells of the skin. The ultraviolet radiation absorbed by the living cells damages the sensitive substances which have an influence on the normal development and aspect of the skin. Damages may cause sunburns, acceleration of the ageing of the skin and/or skin cancer.

Sunburn is the most known and immediate effect of ultraviolet radiation on the skin. This is an inflammation caused by an increase in the blood flow under the skin. A short intense exposure may cause a serious sunburn in persons which are not used to strong sun. It is proved that this type of exposure, as well as long term exposures, may cause the occurrence of skin cancers. Repeated exposure to the sun's ultraviolet radiation also participates in the ageing process. The skin becomes thinner in places and loses its elasticity; imperfections, hyperpigmentation and wrinkles also appear. These changes may occur after many years of exposure but, when they occur, the damages are irreversible. If exposure to the sun continues for several years, the damaged skin incurs a greater risk of developing one of the forms of skin cancer. Exposure to ultraviolet radiation increases the risk of developing these cancers. It seems that discontinuous (occasional) exposure and exposure during childhood and adolescence are probably important predictors of basocellular carcinoma and malignant skin melanoma. High levels of chronic exposure, as in the case of persons working outdoors, are more often associated with spinocellular epithelomia.

Three different types of skin cancer are related to exposure to the sun: basocellular carcinoma; spinocellular epithelioma; and malignant melanoma.

Patent application FR 2 630 648 describes a topical anti-inflammatory or anti-irritating composition comprising a zinc salt of one or more compounds selected from unsaturated fatty acids, polyunsaturated fatty acids and their cyclic derivatives, in a pharmaceutically approved excipient. This application also teaches that the derivatives of certain unsaturated or polyunsaturated fatty acids or protenoic acid may have an anti-inflammatory activity; a possible anti-inflammatory activity of polyunsaturated fatty acids is neither described nor suggested.

International application WO 03/045168 describes formulations based on C18 polyunsaturated fatty acids. These formulations in particular allow the use of these trans/cis conjugated C₁₈ polyunsaturated fatty acids, with their known properties: action on triglycerides or cholesterol or gain of weight, cardiovascular diseases, prevention of cancers, anti-arteriosclerotic properties, prevention or treatments of allergies.

It is known that certain conjugated octadecatrienoic fatty acids are inhibitors of the biosynthesis of prostaglandins (Naturally occurring conjugatedd octadecatrienoic acids are strong inhibitors of prostaglandin biosynthesis; de Nugteren D. H. et al.). However, this article only refers to results obtained in vitro on sheep microsomes. These results cannot predict the in vivo activity of the tested products. Moreover, the tolerance of these products in vivo has not been tested.

The investigation conducted by Nugteren et al., was performed under basal conditions, i.e., without any inflammation. This investigation is not able to predict the activity of the tested products in an inflammatory situation, notably upon applying these products on a skin or inflamed mucosa(s).

Surprisingly, the inventors have discovered that conjugated triene-containing and possibly oxygenated fatty acids of synthetic and/or natural origin may be used in treating the inflammation and/or for promoting healing.

The object of the present invention is therefore the use of a composition comprising at least one fatty acid selected from the group formed by alpha-eleostearic acid, catalpic acid, calendic acid, jacaric acid, licanic acid, and beta-eleostearic acid, for making a drug intended to prevent and/or treat inflammatory diseases and/or metabolic disorders consecutive to inflammation of the skin, of the mucosas and/or cartilage. Alternatively, the composition may comprise alpha-linolenic acid, alone or as a mixture with at least one aforementioned conjugated fatty acid.

Within the scope of the present invention, the expression “metabolic disorders consecutive to inflammation of the skin, of the mucosas and/or cartilages” means all the diseases in relation with a perturbation of metabolism, said perturbation of metabolism being induced or consecutive to an inflammation. These metabolic disorders therefore have in common an inflammation of the skin, of the mucosas, and/or the cartilages and they correspond to the primary or secondary effects of said inflammation.

The mucosas are advantageously selected from the group formed by the buccal, gingival, broncho-pulmonary, auricular, nasal, intestinal, rectal or vaginal mucosas.

Alpha-Eleostaric Acid is Described by the Following Formula:

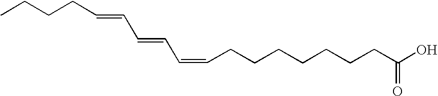

Catalpic Acid is Described by the Following Formula:

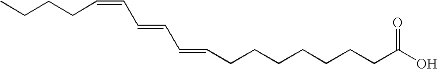

Calendic Acid is Described by the Following Formula:

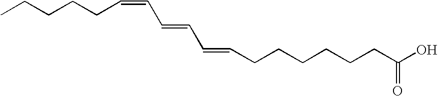

Jacaric Acid is Described by the Following Formula:

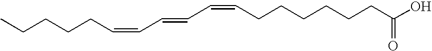

Punicic Acid is Described by the Following Formula:

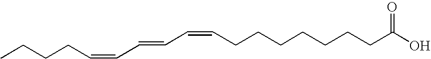

Licanic Acid is Described by the Following Formula:

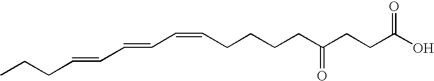

Beta-Eleostearic Acid is Described by the Following Formula:

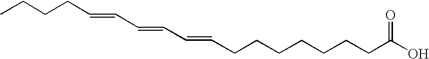

According to an advantageous alternative of the invention, the composition according to the invention comprises at least alpha-eleostearic acid and/or catalpic acid.

According to an advantageous alternative of the invention, the conjugatedd fatty acid is selected from the group formed by alpha-eleostearic acid and catalpic acid, either alone or as a mixture.

Within the scope of the present invention, the inflammation may have a physical (heat, cold, ionizing radiations, infrared radiations, solar radiation), mechanical (friction), chemical (contact with irritating or allergizing products such as perfume or chemical products) or biological origin (microbe, fungus).

According to an alternative of the invention, the inflammation is due to solar radiations, to ionizing radiations, to infrared radiations, to heat or to cold. The drug is then advantageously intended for preventing and/or treating diseases selected from the group formed by skin cancers, solar erythema, and benign summer lucitis. Skin cancers may follow inflammation of the skin due to solar exposure, such as for example a sunburn. Said skin cancers which may be treated within the scope of the present invention, are in particular selected from the group formed by basocellular cancer, spinocellular cancer, or malignant melanoma.

According to another alternative of the invention, the drug is intended for preventing and/or treating allergic and/or irritative reactions of the skin and/or mucosas.

The drug according to the invention is also suitable for promoting healing in normal or pathological healing processes, such as ulcers and eschars.

The drug according to the invention is advantageously intended for preventing and/or treating diseases selected from the group formed by atopical eczema, inflammatory dermatoses such as psoriasis, irritative dermites, acne, seborrheic dermitis, nummular eczema, dyshidrotic eczema, Pityriasis alba, crackled eczema, nutritional eczemas, urticaria, parasite dermatoses, viral dermatoses, fungic or bacterial dermatoses, intertrigo, inflammatory disorders of topical vascularization, foot ulcer and/or insect stings.

Within the scope of the present invention, the drug is advantageously intended for preventing and/or treating diseases selected from the group formed by gingivites and parodontites.

The drug is also advantageously intended for preventing and/or treating diseases selected from the group formed by vulvites and vaginites.

Finally, the drug is also advantageously intended for preventing and/or treating diseases selected from the group formed by arthritis and arthrosis.

Alpha-linolenic acid has already been described as an anti-inflammatory agent (EP 0 226 468). The inventors have discovered that it might be used for making a drug intended to prevent and/or treat inflammatory diseases and/or metabolic disorders consecutive to an inflammation of the skin, mucosas, and/or cartilages. In particular, alpha-linolenic acid may be used for making a drug intended for treating:
skin cancers, solar erythema and benign summer lucitis, in particular basocellular cancer, spinocellular cancer or malignant melanoma;
allergic and/or irritative reactions of the skin and/or mucosas;
upon healing;
diseases selected from the group formed by atopical eczema, inflammatory dermatoses such as psoriasis, irritative dermites, acne, seborrheic dermitis, nummular eczema, dyshidrotic eczema, Pityriasis alba, crackled eczema, nutritional eczemas, urticaria, parasite dermatoses, viral dermatoses, fungic or bacterial dermatoses, intertrigo, inflammatory disorders of topical vascularization, foot ulcer, and/or insect stings;
gingivites and parodontites;
vulvites and vaginites;
arthritis and arthrosis.

The object of the present invention is also the use of a composition comprising at least one fatty acid selected from the group formed by alpha-eleostearic acid, catalpic acid, calendic acid, jacaric acid, licanic acid, and beta-eleostearic acid, for cosmetic treatment of sensitive, irritated, intolerant, allergy-prone, aged skins and/or mucosas, having a disorder of the skin barrier, having cutaneous red spots or having a non-pathological immunological disequilibrium related to intrinsic, extrinsic or hormonal ageing. Alpha-linolenic acid may also be used in a cosmetic composition for the cosmetic treatment of sensitive, irritated, intolerant, allergy-prone, aged, skins and/or mucosas having a disorder of the skin barrier, having cutaneous red spots or having a non-pathological immunological disequilibrium related to intrinsic, extrinsic, or hormonal aging.

Within the scope of the present invention, the cosmetic treatment may consist in applying or ingesting a cosmetic or nutraceutical composition, respectively.

The object of the present invention is also the use of a composition comprising at least one conjugated fatty acid selected from the group formed by alpha-eleostearic acid, catalpic acid, calendic acid, jacaric acid, licanic acid, and beta-eleostearic acid, for cosmetic treatment of cellulitis.

Alpha-linolenic acid may also be used in a cosmetic composition for cosmetic treatment of cellulitis.

Cellulitis is an inflammation of the cell tissue essentially localized under the skin in predisposed regions (thighs, hips, buttocks). Cellulitis corresponds to an inflammation of the subcutaneous cell tissue and appears as an induration of the affected region. This phenomenon may be due to insufficient flow of local microcirculation which alters the exchanges between the blood and the cell tissues, which causes excessive fixation of water, fat, and an accumulation of waste at the cells. This appears as hard adipous masses, “nodes of fat”, which painfully stretch the skin, often at the same places: inside and/or outside the thighs, on the hips, the buttocks, the stomach, and the chest, and sometimes on the arms and on the nape. The presence of cellulitis may not be related to the weight of the person.

Within the scope of the present invention, the composition comprising at least one conjugated fatty acid selected from the group formed by alpha-eleostearic acid, catalpic acid, calendic acid, jacaric acid, licanic acid, and beta-eleostearic acid may be formulated as different preparations suitable for topical, oral, rectal, vaginal, nasal, auricular, bronchial, or parenteral administration. The composition according to the invention is advantageously formulated for an administration via a topical or oral route. Alternatively, the composition may comprise alpha-linolenic acid, alone or as a mixture with at least one aforementioned conjugated fatty acid.

When the composition is suitable for administration via a topical route, the different formulations may include creams, ointments, lotions, oils, patches, sprays or any other products for external application.

The composition intended to be administered via a topical route advantageously comprises 0.001 to 50% by weight, even more advantageously 0.5 to 20% by weight, of at least one conjugated fatty acid selected from the group formed by x-eleostearic acid, catalpic acid, calendic acid, jacaric acid, licanic acid, and β-eleostearic acid, based on the total weight of said composition.

According to an advantageous alternative of the invention, the composition administered via a topical route comprises 0.001 to 50% by weight, advantageously 0.5 to 20% by weight, even more advantageously 1 to 10% by weight of alpha-eleostearic acid, and 0.001 to 50% by weight, advantageously 0.5 to 20% by weight, even more advantageously 1 to 10% by weight, of catalpic acid, based on the total weight of said composition.

According to another alternative of the invention, the composition administered via a topical route, advantageously comprises 0.001 to 50% by weight, advantageously 0.5 to 20% by weight, even more advantageously 1 to 10% by weight, of alpha-eleostearic acid, based on the total weight of said composition.

When the drug or the cosmetic composition is administered per os, it may be administered as unit dosage forms or multi-doses for administration as a mixture with adequate pharmaceutical or cosmetic carriers known to one skilled in the art. The suitable unit dosage forms notably comprise possibly scored tablets, gelatine capsules, powders, granules and oral solutions or suspensions. The suitable multi-dose dosage forms notably comprise oral drops, emulsions and syrups. The composition may be formulated as a food supplement.

During the preparation of tablets, the conjugated fatty acid(s) according to the invention are mixed with a pharmaceutically or cosmetically acceptable vehicle such as notably gelatine, talc, starch, lactose, magnesium stearate acacia gum or their analogs. The tablets may possibly be coated, i.e., covered with several layers of different substances such as saccharose, in order to facilitate intake or preservation. The tablets may further have a more or less complex formulation intended to change the release rate of the active ingredient. Release of the active ingredient of said tablet may be accelerated, slowed down or delayed depending on the desired absorption.

A gelatine capsule preparation is obtained by mixing the conjugated fatty acid(s) according to the invention with a diluent. The thereby obtained mixture is poured into soft or hard gelatine capsules. A preparation as a syrup may contain the conjugated fatty acid(s) according to the invention together with a sweetener, advantageously an acaloric sweetener, a gustatory agent and a suitable colouring agent. The powders or granules dispersible in water may comprise the conjugated fatty acid(s) according to the invention as a mixture with dispersants or wetting agents, suspension agents, such as polyvinylpyrrolidone or sweeteners or taste correcting agents.

The orally administered composition advantageously comprises 0.001% to 100% by weight, even more advantageously 1 to 50% by weight, of at least one conjugated fatty acid selected from the group formed by α-eleostearic acid, catalpic acid, calendic acid, jacaric acid, licanic acid and β-eleostearic acid, based on the total weight of said composition.

According to an advantageous alternative of the invention, the orally administered composition comprises 0.001 to 100% by weight, advantageously 1 to 50% by weight, of x-eleostearic acid, and 0.001 to 100% by weight, advantageously 1 to 50% by weight of catalpic acid, based on the total weight of said composition.

According to another alternative of the invention, the orally administered composition comprises 0.001 to 100% by weight, advantageously 1 to 50% by weight, of x-eleostearic acid, based on the total weight of said composition.

When the composition according to the invention is formulated as a food supplement, said food supplement may comprise up to 100% by weight of active ingredients according to the invention, i.e., particular fatty acids defined in the present invention.

The amount of active ingredients which the composition according to the invention will comprise, mainly depends on the method of administration and of the selected dosage form.

The methods of administration, the dosages, and the optimum dosage forms of the compositions according to the invention may be determined according to criteria generally taken into account upon establishing a pharmaceutical treatment, in particular a dermatological or cosmetic treatment, suitable for a patient, such as for example the age and body weight of the patient, the seriousness of his/her general condition, the tolerance to the treatment, the reported secondary effect, the type of skin. Depending on the type of desired administration, the drug and/or the active compounds according to the invention may further comprise at least one pharmaceutically acceptable excipient, notably dermatologically acceptable or cosmetically acceptable excipient. Preferably an excipient is used which is adapted to administration via an external topical or oral route. The drug or the cosmetic composition according to the present invention may further comprise at least one adjuvant, pharmaceutically or cosmetically known to one skilled in the art, selected from thickeners, preservatives, perfumes, colouring agents, chemical or mineral filters, moisturizing agents, thermal waters, etc. The drug according to the invention is intended for treating and/or preventing diseases which may affect the human being and/or animals, notably mammals.

The drug or cosmetic composition according to the invention may further comprise, in association, advantageously with a synergistic effect, at least one compound selected from the group formed by emollients, moisturizing actives, promoters of keratin synthesis, kerato-regulating agents, keratolytic agents, agents for restructuring the skin barrier (promoters of synthesis of cutaneous lipids), keratinocyte differentiation promoters (retinoids or retinoid-like Calcidone®, calcium), epidermis differentiation modulating agents, agents for consolidating the dermo-epidermal junction, antibiotics, anti-bacterial agents, anti-fungic compounds, anti-viral agents, stimulators of innate immunity (natural antibiotic peptides) or of acquired immunity, sebo-regulating agents such as the inhibitors of 5-alpha reductase, notably the 5-alpha active Avocuta® marketed by Laboratoires Expanscience or zinc salts and sabal (Sabalinae of the Coryphoideae sub-family), immunomodulators, such as tacrolimus, pimecrolimus, oxazolines, preservatives, anti-irritation agents, soothing agents, sun filters and screens, anti-oxidants, growth factors, healing agents or eutrophic molecules, drugs and anti-inflammatory agents, and compounds containing unsaponifiables of vegetable oils.

According to an advantageous alternative of the invention, the composition comprises in association, advantageously with a synergistic effect, active ingredients selected from the group formed by anti-inflammatory agents.

Promoters of keratin synthesis which may be used in association, advantageously with a synergistic effect, with conjugated fatty acids, advantageously are retinoids or retinoids like peptides marketed by Silab, key-proteins of the stratum corneum or granulosum (keratins).

Antibiotics which may be used in association, advantageously with a synergistic effect, with the conjugated fatty acids advantageously are fucidic acid, penicillin, tetracyclins, pristinamycin, erythromycin, clindamycin, mupirocin, minocyclin, doxycyclin. Antiviral agents which may be used in association with the conjugated fatty acids are acyclovir and valacyclovir advantageously.

The anti-irritation agents which may be used within the scope of the present invention, in association, advantageously with a synergistic effect, with conjugated fatty acids advantageously are glycine, lupin sugars and/or peptides, avocado sugars and/or peptides, as described in patent applications FR 0404635 and FR 0404640, Cycloceramide®.

Soothing agents which may be used in association, advantageously with a synergistic effect, with the conjugated fatty acids, advantageously are alpha-bisabolol, liquorice derivatives, enoxolone (3-beta-hydroxy-11-oxo-olean-12-en-30-oic acid, CAS number 471-53-4). Kerato-regulating agents which may be used in association with the conjugated fatty acids advantageously are alpha-hydroxyacids and their derivatives. Keratolytic agents which may notably be used in association with the conjugated fatty acids are salicylic acid and its derivatives: lipohydroxyacids. Anti-oxidants which may be used in the invention in association, with conjugated fatty acids advantageously are vitamins (C, E), trace elements (copper, zinc, and selenium), anti-oxidant enzymes. Growth factors which may be use in association, advantageously with a synergistic effect, with the conjugated fatty acids, advantageously are becaplermin and beta-TGF (beta Transforming Growth Factor).

Healing agents which may be used in association, advantageously with a synergistic effect, with the conjugated fatty acids, advantageously are vitamin A, panthenol, Avocadofurane®, zinc oxide, magnesium, silicon, madecassic or asiatic acid, polysaccharides of all origins and in particular of marine thermal origin or derivatives of pearl oysters.

Drugs which may be used within the scope of the present invention, in association, advantageously with a synergistic effect, with the conjugated fatty acids, advantageously are drugs, suitable for topical or oral administration, for preventing and/or treating atopy (corticoids, emollients, immunomodulators), acne (antibiotics, benzoyl peroxide, topical and oral retinoids, azelaic acid, vitamin PP, zinc, cyclins), eczema (immunomodulators, emollients, fish oil, borage oil, pre- and pro-biotics) or psoriasis (corticoids, calcipotriol, calcitriol, tazarotene, cade oil, acitretin, PUVA therapy, derivatives of vitamin D). The drug may also comprise in association, stimulators of antibiotic peptides such as avocado sugars and avocado peptides.

Anti-inflammatory agents which may be used in association, advantageously with a synergistic effect, with the conjugated fatty acids, advantageously are steroid anti-inflammatory agents (AIS), such as corticoids or non-steroids (AINS) and anti Cox 2 (celecoxib).

Agents for restructuring the skin barrier, with which synthesis of the key lipids of the epidermis may be stimulated, and which may be used in association, advantageously with a synergistic effect, with the conjugated fatty acids, advantageously are sunflower concentrates, even more advantageously linoleic sunflower concentrates, such as the active marketed by Latoratoires Expanscience, Soline® (cf. International Application WO 01/21150), unsaponifiables of vegetable oil, such as Avocadofurane® (cf. International Application WO 01/21150), alpha, beta, gamma and delta PPAR agonists (rosiglitazone, pioglitazone), RXR and RAR agonists of vitamin D receptors. Antifungic compounds which may be used in association with the conjugated fatty acids advantageously are econazole, ketoconazole and climbazole. Reducing agents such as ichtyol may also be used in association.

Antiseptic preservatives which may be used in association, advantageously with a synergistic effect, with the conjugated fatty acids, may be triclosan, chlorhexidin, quaternary ammoniums.

Immunomodulators which may be used in association, advantageously with a synergistic effect, with the conjugated fatty acids advantageously are tacrolimus, pimecrolimus and oxazolines.

Oxazolines which may be used within the scope of the present invention advantageously are oxazolines selected from the group formed by 2-undecyl-4-hydroxymethyl-4-methyl-1,3-oxazoline, 2-undecyl-4,4-dimethyl-1,3-oxazoline, (E)-4,4-dimethyl-2-heptadec-8-enyl-1,3-oxazoline, 4-hydroxymethyl-4-methyl-2-hepta-decyl-1,3-oxazoline, (E)-4-hydroxymethyl-4-methyl-2-heptadec-8-enyl-1,3-oxazoline, 2-undecyl-4-ethyl-4-hydroxymethyl-1,3-oxazoline. Even more advantageously, said oxazoline is 2-undecyl-4,4-dimethyl-1,3-oxazoline, called OX-100 or Cycloceramide®.

Compounds containing unsaponifiables of vegetable oils which may be used in association, advantageously with a synergistic effect, with the conjugated fatty acids, are advantageously selected from the group formed by avocado furane lipids, avocado and soya unsaponifiables, lupine oil concentrates, sunflower oil concentrates and mixtures thereof. The avocado furane lipids which may be used within the scope of the present invention advantageously are natural 2-alkylfuranes, notably the active Avocadofurane®, marketed by Laboratoires Expanscience, which may be obtained by the method described in International Application WO 01/21605. The avocado and soya unsaponifiables which may be used in association with the conjugated fatty acids advantageously are a mixture of furane avocado unsaponifiables and of soya unsaponifiables, in a respective ratio of about 1/3 to 2/3. The avocado and soya unsaponifiables even more advantageously are the product Piascledine®, marketed by Laboratoires Expanscience. Lupin oil concentrates which may advantageously be used, are concentrates obtained by molecular distillation of lupin oil, advantageously of mild white lupin oil, such as those described in International Application WO 98/47479. Advantageously they contain about 60% by weight of unsaponifiables. Sunflower oil concentrates which may be used advantageously are linoleic sunflower concentrates, such as the active Soline® marketed by Laboratoires Expanscience (cf. International Application WO 01/21150).

According to an advantageous alternative of the invention, the conjugated fatty acid source is a lipid extract of at least one plant selected from the group formed by plants of the Cucurbitaceae, Punicaceae, Bignoniaceae, Euphorbiaceae, Compositeae (Asteraceae), Balsaminaceae, Rosaceae, Chrysobalanaceae, Ricinocarpus and Chilopsis family. More particularly, the conjugated fatty acid source is a lipid extract of at least one plant selected from the group formed by green, white, pearl and wild Mormordica, Catalpa, Aleurites, Euphorbia, Parinarium, Licania, Parinarium, Calendula, Punica, pomegranate tree, China wood, balsam, Trichosanthes, Centratus, and Jacaranda.

The source of alpha-eleostearic acid advantageously is virgin and/or refined oils of at least a plant selected from the group formed by Momordica charantia, Aleurites montana and Aleurites fordii, Parinarium montanum, Parinarium excelsum, Parinarium macrophyllum, Parinarium holstil, Licania rigida and Ricinocarpus bowmanii. The source of catalpic acid advantageously is virgin and/or refined oils of at least a plant selected from the group formed by Catalpa ovata, Catalpa bignoniodes and Chilopsis linearis. The source of calandic acid advantageously is a virgin and purified oil of Calendula officinalis. The source of punicic acid advantageously is virgin and/or refined oils of at least one plant selected from the group formed by Punica granatum, Trichosanthes nervifolia, and Momordica balsamina. The source of beta-eleostearic acid advantageously is a virgin and purified oil of Centratus ruber. The source of licanic acid advantageously is oiticica, licania or parinarium oil. According to an advantageous alternative of the invention, the aforementioned virgin and/or refined oils do not contain any trace of detectable protein.

The source of alpha-eleostearic acid and of catalpic acid more particularly is a lipid extract from Mormodica seeds, advantageously from Momordica charantia seeds.

According to an advantageous alternative of the invention, the topically administered composition according to the invention comprises 0.001% to 50% by weight of a lipid extract of Momordica seeds, advantageously 0.001% to 50% by weight, even more advantageously 2 to 20% by weight of a lipid extract of Momordica charantia seeds, based on the total weight of the composition.

According to another advantageous alternative of the invention, the orally administered composition comprises 0.001% to 100% by weight of a lipid extract of Momordica seeds, advantageously 0.001% to 100% by weight of lipid extract of Momordica charantia seeds, even more advantageously 1 to 50% by weight of a lipid extract of Momordica charantia seeds, based on the total weight of the composition.

The lipid extract of Momordica charantia seeds advantageously comprises at least 20% by weight of alpha-eleostearic acid and catalpic acid, advantageously at least 40% by weight of alpha-eleostearic acid and catalpic acid, even more advantageously about 45% by weight of alpha-eleostearic acid and catalpic acid. The mixture of alpha-eleostearic acid and catalpic acid advantageously comprises at least 50% by weight of alpha-eleostearic acid, advantageously at least 90% by weight, of alpha-eleostearic acid, even more advantageously at least 98% by weight of alpha-eleostearic acid.

Margoze of the Momordica charantia genus is a tropical Cucurbitacea which comes from India, but which is also grown on Reunion Island. This is an annual Herbacea which may provide 2 meter long lianas, and the green coloured oblong fruit of which contains flat seeds. The lipid content of these seeds is about 30% by weight. The lipid extract of Momordica charantia may be obtained by a method consisting of extracting total lipids from the seeds of Momordica charantia, dried and milled beforehand, by means of an oil solvent, and then evaporating said solvent or according to the method consisting of extracting the lipids from the seeds of Momordica charantia by mechanical cold-pressing of the seeds.

In a particular embodiment according to the present invention, the oil from the seeds of Momordica charantia may be obtained according to the method consisting of extracting the total lipids of the Cucurbitacea seeds, dried and milled beforehand, by means of an oil solvent, and then evaporating the solvent. The seeds of Momordica charantia according to the present invention are for example milled by means of cylinder or hammer mill. The oil solvent, used for extracting the total lipids from the seeds forming the oil is a conventional organic solvent for extracting lipids. The solvent is advantageously selected from the group formed by aliphatic alkanes, aromatic alkanes, aliphatic alcohols, and their halogenated derivatives. Even more advantageously according to the present invention, the organic solvent is hexane. Extraction of the total lipids from seeds of Momordica charantia is advantageously performed by soxhlet extraction which is a technology well known to one skilled in the art. After extracting the lipids contained in the seeds of Momordica charantia according to the present invention, the organic solvent is evaporated, preferably by evaporation in vacuo.

In another particular embodiment according to the present invention, the oil from the seeds of Momordica charantia may be obtained according to the method consisting of extracting the lipids from Cucurbitacea seeds, by mechanical cold pressing of the seeds, adavantageously by means of a continuous screw press in order to lead after filtration, to first-pressing virgin oils.

The lipid extracts of other plants according to the present invention may be obtained by methods similar to the methods described above. The oils of the plant(s) according to the present invention may be used either crude or refined. Refining in the sense of the present invention, means the unitary operations for purifying lipids of plant origin well known to one skilled in the art, among which chemical neutralisation, demucilagination, discoloration, deodorization, and frigelisation may notably be mentioned. Oils extracted from seeds of Momordica charantia have the additional advantage of being cosmetically acceptable compounds, non-aggressive to the skin, non-toxic and hypoallergenic.

The following examples are given as non-limiting examples and illustrate the present invention.

### EXAMPLE 1

### Cosmetic Formulations Based on an Extract of Momordica charantia

| | |
|---|---|
| | Anti-acne cream No. 1 |
| Water QSP | 100% |
| Isononyl isononoate | 7.000 |
| Di-C₁₂-C₁₃ alkyl malate | 7.000 |
| Isocetyl stearate | 5.000 |
| Butylene glycol | 3.000 |
| Oriza sativa | 2.500 |
| Momordica charantia extract | 2.000 |
| Dicaprylyl ether | 2.000 |
| Silanediol salicylate | 2.000 |
| Arachic alcohol | 1.650 |
| Tromethamine | 1.180 |
| Cetyl alcohol | 1.000 |
| Salicylic acid | 1.000 |
| Glucoside ascorbyl | 1.000 |
| Glycine | 1.000 |
| Tocopheryl acetate | 1.000 |
| Behenyl alcohol | 0.900 |
| Squalane | 0.790 |
| Sodium citrate | 0.660 |
| Copolymer PPG-12/SMDI | 0.500 |
| Glucoside arachidyl | 0.450 |
| Perfume | 0.400 |
| Sclerotium gum | 0.160 |
| Cetearyl alcohol | 0.130 |
| Citric acid | 0.110 |
| Sepigel 305* | 0.100 |
| Preservative system | QS |
| | Anti-acne cream No. 2 |
| Water QSP | 100% |
| Isononyl isononoate | 7.000 |
| Di-C₁₂-C₁₃ alkyl malate | 7.000 |
| Isocetyl stearate | 5.000 |
| Butylene glycol | 3.000 |
| Oriza sativa | 2.500 |
| Momordica charantia extract | 2.000 |
| 5-alpha avocuta ® | 2.000 |
| Dicaprylyl ether | 2.000 |
| Silanediol salicylate | 2.000 |
| Arachic alcohol | 1.650 |
| Tromethamine | 1.180 |
| Cetyl alcohol | 1.000 |
| Salicylic acid | 1.000 |
| Glucoside ascorbyl | 1.000 |
| Glycine | 1.000 |
| Tocopheryl acetate | 1.000 |
| Behenyl alcohol | 0.900 |
| Squalane | 0.790 |
| Sodium citrate | 0.660 |
| Copolymer PPG-12/SMDI | 0.500 |
| Glucoside arachidyl | 0.450 |
| Perfume | 0.400 |
| Sclerotium gum | 0.160 |
| Cetearyl alcohol | 0.130 |
| Citric acid | 0.110 |
| Sepigel 305* | 0.100 |
| Preservative system | QS |
| | Mouth wash |
| Momordica charantia extract | 0.1-10% |
| Ethyl alcohol | 10 |
| Glycerine | 10 |
| Hydrogenated castor oil, | 0.5 |
| ethoxylated with 40 moles of EO | |
| (Cremophor co410) | |
| Poly(methylvinylether/maleic acid) | 0.2 |
| (Gantrez S97BF) | |
| Soda | 0.15 |
| Sodium fluoride | 0.05 |
| Cinnamon/mint aroma | 0.1 |
| Triclosan | 0.03 |
| Zinc chloride | 0.01 |
| Sodium saccharine | 0.01 |
| Colouring C.I. 16255 (E124) | 0.0025 |
| Purified water QSP | 100 |
| | Anti-cellulitis cream gel (%) |
| Water QSP | 100 |
| Cyclomethicone | 5.40 |
| Octyl palmitate | 5.00 |
| Hydrogenated coco-glycerides | 3.00 |
| Behenyl alcohol from ground nut | 2.55 |
| Propylene glycol | 2.50 |
| Isodecyl neopentanoate | 2.00 |
| Glyceryl stearate | 1.70 |
| Cetyl alcohol | 1.30 |
| Stearic acid | 1.00 |
| PEG-6 | 1.00 |
| Beeswax | 0.40 |
| C₁₃-C₁₄ isoparaffin | 0.40 |
| Butylene glycol | 0.16 |
| Glycerine | 0.16 |
| Cetearyl alcohol | 0.10 |
| Cetyl palmitate | 0.10 |
| Cocoglycerides | 0.10 |
| Laureth-7 | 0.10 |
| 4,5,7-trihydroxyisoflavone | 0.01-10 |
| Enteromorpha Compressa Extract | 0.01-5 |
| Sophora Japonica Extract | 0.01-20 |
| Centella Asiatica Extract | 0.01-5 |
| Momordica charantia Extract | 0.01-10 |
| Preservative QS | |
| Perfume QS | |

*Product marketed by Seppic
QSP: Quantum satis pro; QS: Quantum satis

### EXAMPLE 2

### Cosmetic Formulations Based on Pomegranate Tree Extract

| | |
|---|---|
| | Anti-acne cream |
| Water QSP | 100% |
| Isononyl isononoate | 7.000 |
| Di-C₁₂-C₁₃ alkyl malate | 7.000 |
| Isocetyl stearate | 5.000 |
| Butylene glycol | 3.000 |
| Oriza sativa | 2.500 |
| Pomegranate tree extract | 2.000 |
| Dicaprylyl ether | 2.000 |
| Silanediol salicylate | 2.000 |
| Arachic alcohol | 1.650 |
| Tromethamine | 1.180 |
| Cetyl alcohol | 1.000 |
| Salicylic acid | 1.000 |
| Glucoside ascorbyl | 1.000 |
| Glycine | 1.000 |
| Tocopheryl acetate | 1.000 |
| Behenyl alcohol | 0.900 |
| Squalane | 0.790 |
| Sodium citrate | 0.660 |
| Copolymer PPG-12/SMDI | 0.500 |
| Glucoside arachidyl | 0.450 |
| Perfume | 0.400 |
| Sclerotium gum | 0.160 |
| Cetearyl alcohol | 0.130 |
| Citric acid | 0.110 |
| Sepigel 305* | 0.100 |
| Preservative system | QS |
| | Mouth wash |
| Pomegranate tree extract | 0.1-10% |
| Ethyl alcohol | 10 |
| Glycerine | 10 |
| Hydrogenated castor oil, | 0.5 |
| ethoxylated with 40 moles of EO | |
| (Cremophor co410) | |
| Poly(methylvinylether/maleic acid) | 0.2 |
| (Gantrez S97BF) | |
| Soda | 0.15 |
| Sodium fluoride | 0.05 |
| Cinnamon/mint aroma | 0.1 |
| Triclosan | 0.03 |
| Zinc chloride | 0.01 |
| Sodium saccharine | 0.01 |
| Colouring C.I. 16255 (E124) | 0.0025 |
| Purified water | QSP 100 |
| | Anti-cellulitis cream gel (%) |
| Water QSP | 100 |
| Cyclomethicone | 5.40 |
| Octyl palmitate | 5.00 |
| Hydrogenated cocoglycerides | 3.00 |
| Behenyl alcohol from ground nut | 2.55 |
| Propylene glycol | 2.50 |
| Isodecyl neopentanoate | 2.00 |
| Glyceryl stearate | 1.70 |
| Cetyl alcohol | 1.30 |
| Stearic acid | 1.00 |
| PEG-6 | 1.00 |
| Beeswax | 0.40 |
| C₁₃-C₁₄ isoparaffin | 0.40 |
| Butylene glycol | 0.16 |
| Glycerine | 0.16 |
| Cetearyl alcohol | 0.10 |
| Cetyl palmitate | 0.10 |
| Cocoglycerides | 0.10 |
| Laureth-7 | 0.10 |
| 4,5,7-trihydroxyisoflavone | 0.01-10 |
| Enteromorpha Compressa extract | 0.01-5 |
| Sophora Japonica extract | 0.01-20 |
| Centella Asiatica extract | 0.01-5 |
| Pomegranate tree extract | 0.01-10 |
| Preservative | QS |
| Perfume | QS |

*Product marketed by Seppic
QSP: Quantum satis pro; QS: Quantum satis

### EXAMPLE 3

### Cosmetic Formulations Based on an Extract of calendula

| | |
|---|---|
| | Anti-acne cream |
| Water | QSP 100% |
| Isononyl isononoate | 7.000 |
| Di-C₁₂-₁₃ alkyl malate | 7.000 |
| Isocetyl stearate | 5.000 |
| Butylene glycol | 3.000 |
| Oriza sativa | 2.500 |
| Extract of calendula | 2.000 |
| Dicaprylyl ether | 2.000 |
| Silanediol salicylate | 2.000 |
| Arachic alcohol | 1.650 |
| Tromethamine | 1.180 |
| Cetyl alcohol | 1.000 |
| Salicylic acid | 1.000 |
| Glucoside ascorbyl | 1.000 |
| Glycine | 1.000 |
| Tocopheryl acetate | 1.000 |
| Behenyl alcohol | 0.900 |
| Squalane | 0.790 |
| Sodium citrate | 0.660 |
| Copolymer PPG-12/SMDI | 0.500 |
| Glucoside arachidyl | 0.450 |
| Perfume | 0.400 |
| Sclerotium gum | 0.160 |
| Cetearyl alcohol | 0.130 |
| Citric acid | 0.110 |
| Sepigel 305* | 0.100 |
| Preservative system | QS |
| | Mouth wash |
| Extract of calendula | 0.1-10% |
| Ethyl alcohol | 10 |
| Glycerine | 10 |
| Hydrogenated castor oil, | 0.5 |
| ethoxylated with 40 moles of EO | |
| (Cremophor co410) | |
| Poly(methylvinylether/maleic acid) | 0.2 |
| (Gantrez S97BF) | |
| Soda | 0.15 |
| Sodium fluoride | 0.05 |
| Cinnamon/mint aroma | 0.1 |
| Triclosan | 0.03 |
| Zinc chloride | 0.01 |
| Sodium saccharine | 0.01 |
| Colouring C.I. 16255 (E124) | 0.0025 |
| Purified water QSP | 100 |
| | Anti-cellulitis cream gel (%) |
| Water | QSP 100 |
| Cyclomethicone | 5.40 |
| Octyl palmitate | 5.00 |
| Hydrogenated cocoglycerides | 3.00 |
| Behenyl alcohol from ground nut | 2.55 |
| Propylene glycol | 2.50 |
| Isodecyl neopentanoate | 2.00 |
| Glyceryl stearate | 1.70 |
| Cetyl alcohol | 1.30 |
| Stearic acid | 1.00 |
| PEG-6 | 1.00 |
| Beeswax | 0.40 |
| C₁₃-₁₄ isoparaffin | 0.40 |
| Butylene glycol | 0.16 |
| Glycerine | 0.16 |
| Cetearyl alcohol | 0.10 |
| Cetyl palmitate | 0.10 |
| Cocoglycerides | 0.10 |
| Laureth-7 | 0.10 |
| 4,5,7-trihydroxyisoflavone | 0.01-10 |
| Extract of Enteromorpha Compressa | 0.01-5 |
| Extract of Sophora Japonica | 0.01-20 |
| Extract of Centella Asiatica | 0.01-5 |
| Extract of Calendula | 0.01-10 |
| Preservative | QS |
| Perfume | QS |

*Product marketed by Seppic
QSP: Quantum satis pro; QS: Quantum satis

### EXAMPLE 4

Study of the effect of an extract of Momordica charantia in a mechanically induced skin inflammation model (shaving) in wild Balb/c mice

Protocol

The study was conducted on 6 week old female mice Balb/c. Each group of mice consisted of 8 mice. On D0, the mice were shaved over the back (surface of about 2 cm²). On D2, a first group of mice is used for obtaining base values. Topical application of an extract of Momordica charantia, in solution in an acetone/olive oil mixture (ratio 4/1), starts on D2. The extract of Momordica charantia is applied at 2% dosage, with two applications per day. On D4, i.e., after 2 days of topical application of Momordica charantia extract, skin biopsies were carried out. A histological analysis is conducted by staining cuts with hematoxylin/eosin.

The Momordica charantia extract used comprises by weight:

| | | |
|---|---|---|
| Unsaponifiables | 0.9% | by weight |
| Stearics | 33.3% | by weight |
| Oleic acid | 3.2% | by weight |
| Linoleic acid | 4.3% | by weight |
| Linolenic acid | >0.1% | by weight |
| Catalpic and/or alpha-eleostearic acid | 49.1% | by weight |

Results

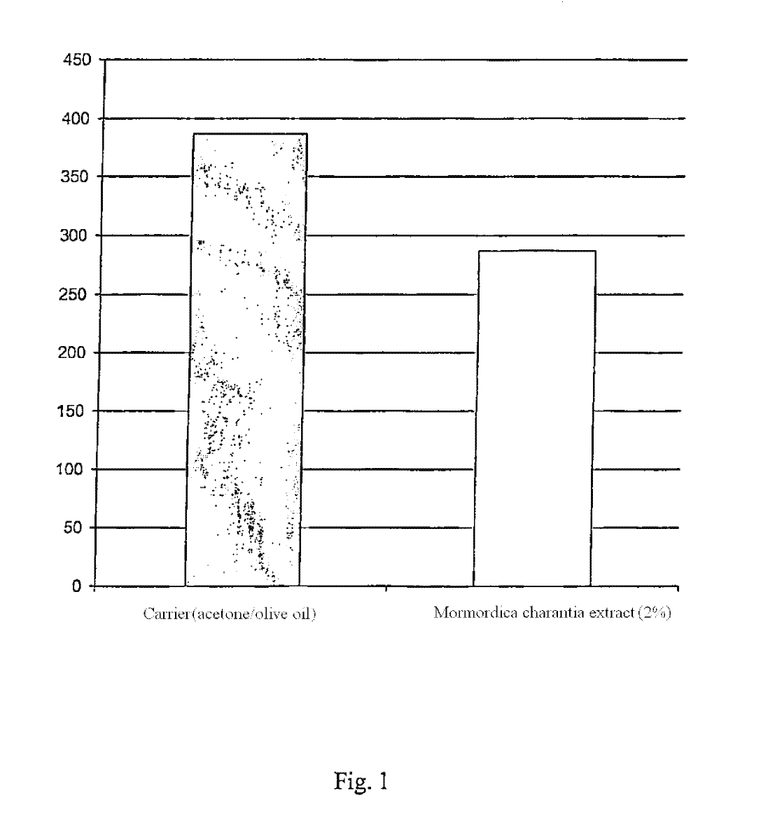

On D4, the animals have the signs of inflammation at the shaving site, which are quantitated via measurement of the thickness of the skin. Indeed, an increase in the thickness of the skin is correlated with inflammation. The group treated with the vehicle (acetone/olive oil) has on D4, a thickness of the skin, statistically larger than that of the group treated with 2% Momordica charantia extract for 2 days (FIG. 1). In other words, it was shown in a quite unexpected way, that a Momordica charantia extract has anti-inflammatory properties in a mechanically induced skin inflammation model in mice. The results are summarized in FIG. 1 enclosed, which illustrates in ordinates, the thickness of the skin in microns on D4, measured in each of the treated groups (vehicle: acetone/olive oil or Momordica charantia extract).

### EXAMPLE 5

Study of the effect of Momordica charantia extract, applied topically at 2% dosage to wild Balb/c mice, on the proliferation of keratinocytes

Protocol

The study was carried out on 6 week old female mice Balb/c. Each group of mice consisted of 8 mice. On DO, the mice are shaved over the back (surface of about 2 cm²). On D2, a first group of mice is used for obtaining the base values. Topical application of a Momordica charantia extract, in solution in an acetone/olive oil (ratio 4/1) mixture, starts on D2. The Momordica charantia extract is applied at 2% dosage for 30 days, with two applications per day. The Momordica charantia extract used is the same at the one of Example 4.

After 4 days of application, skin biopsies are carried out and analyzed by immuno-histochemistry with an anti-Ki67 antibody (proliferation marker). Five different measurements are carried out for each of the mice.

Results

As shown in the enclosed figure, topical application of a Momordica charantia extract at 2% dosage (for 4 weeks, with two applications per day), causes a statistically significant increase in the percentage of Ki-67 positive cells with respect to the vehicle alone.

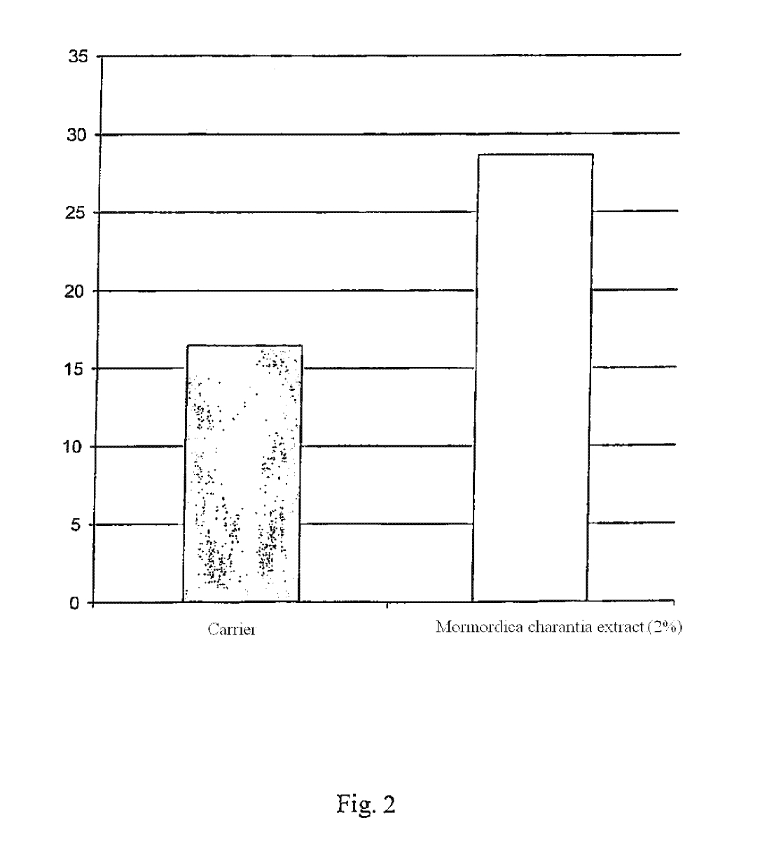

FIG. 2 enclosed illustrates in ordinates the percentage of positive Ki-67 cells versus the total number of cells measured on D30 in each of the treated groups (vehicle: acetone/olive oil or Momordica charantia extract).

It is thus shown that quite surprisingly, a Momordica charantia extract causes an increase in the proliferation in vivo of keratinocytes in mice. In other words, the Momordica charantia extract has healing and eutrophic properties. So-called eutrophic properties are notably advantageous within the scope of skin pathologies characterized by a disorder of the skin barrier.

### EXAMPLE 6

### Evaluation of Lipid Synthesis in Suspended Adipocytes

Tested Product

The Momordica charantia extract used is the same as that of Example 4.

Culture Conditions

Normal human adipocytes were isolated from abdominal biopsies (plastic surgery). Immediately after receipt, the samples were incubated for 30 min at 37° C. in the presence of collagenase (Sigma). The suspension of adipocytes is then rinsed and diluted 3 times in the culture medium.

Culture medium: bicarbonate (Life Technologies) 1.87 mg/ml, penicillin/streptomycin (Life Technologies) 25 IU/ml/25 μg/ml, glutamine (Life Technologies) 2 mM, MEM (Merck Eurolab) 100% v/v, albumin of bovine origin (Sigma) 0.5% w/v.

Evaluation of Lipid Synthesis:

The suspended adipocytes are incubated for 1 h at 37° C. in the presence of different concentrations of MA105 (20 and 2 ppm) diluted in THF (tetrahydrofurane). A 10 ml volume of radio-labelled acetate (2-C¹⁴, 60.87 μCi/ml, Amersham) is then added to the preparation. After 4 hrs of incubation, the lipids are extracted according to the procedure described by Bligh and Dyer (methanol/chloroform/water), evaporated under nitrogen and the incorporated radioactivity was quantitated by liquid scintillation (LKB 1210 Rackbeta).

Results:

The Momordica charantia extract does not induce any interference with the radio-labelling. The reference molecule, ceruline (inhibitor of FAS, Fatty Acid Synthase) tested at 10 μM inhibits the incorporation of acetate (75% inhibition/control). This result validates the test. The Momordica charantia extract tested at 2 and 20 ppm significantly reduces the incorporation of acetate in lipids (25 and 30% of the control, respectively). The Momordica charantia extract shows a significant inhibitory activity of lipid synthesis. The Momordica charantia extract is therefore capable of inhibiting lipogenesis in human adipocytes in culture. This experiment quite surprisingly shows the benefit obtained from the product in treating cellulitis.

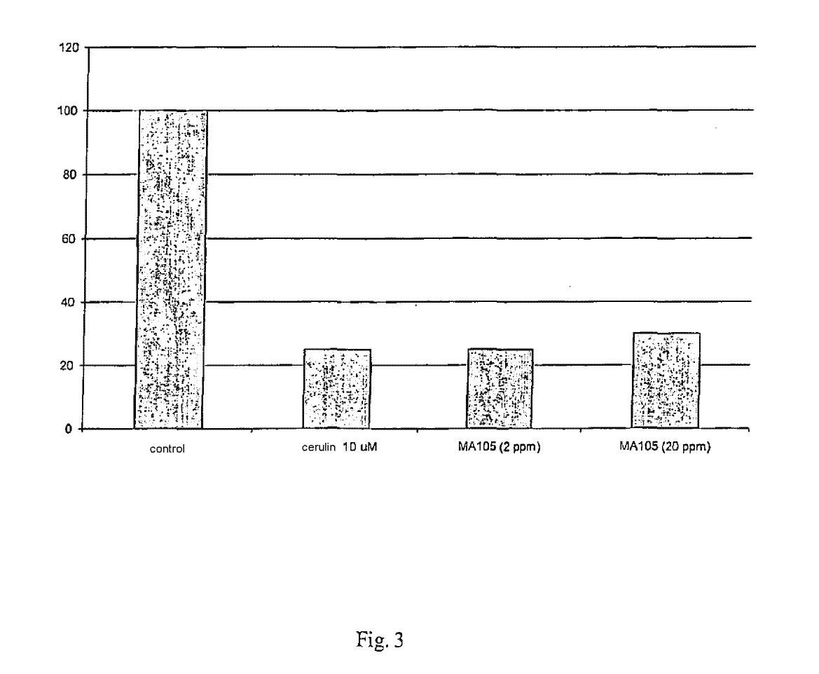

FIG. 3 enclosed illustrates the effect of Momordica charantia extract on the incorporation of radio-labelled acetate in adipocyte lipids. The results are significative (p<0.01).

1-21. (canceled)

22. A method for preventing or treating inflammation comprising the administration of a composition comprising an effective amount of at least one fatty acid selected from the group formed by alpha-eleostearic acid, catalpic acid, calendic acid, jacaric acid, licanic acid, and beta-eleostearic acid, to a patient in need thereof.

23. the method according to claim 22, wherein the inflammation prevented or treated is selected from the group consisting of:
inflammatory diseases or metabolic disorders consecutive to inflammation of the skin, the mucosas, and/or the cartilages;
inflammation due to solar radiations, ionizing radiations, infrared radiations, heat or cold;
allergic and/or irritative reactions of the skin and/or the mucosas;
atopical eczema, inflammatory dermatoses, irritative dermites, acne, seborrheic dermitis, nummular eczema, dyshidrotic eczema, pityriasis alba, crackled eczema, nutritional eczema, urticaria, parasite dermatoses, viral dermatoses, fungic or bacterial dermatoses, intertrigo, inflammatory disorders of topical vascularization, foot ulcer and/or insect stings;
sensitive, irritated, intolerant, allergy-prone, aged, skins and/or mucosas, having a disorder of the skin barrier, having cutaneous red spots or having an immunological non-pathological disequilibrium related to intrinsic, extrinsic or hormonal ageing; and
cellulitis.

24. (canceled)

25. The method according to claim 23, wherein the inflammatory diseases are selected from the group formed by skin cancers, solar erythema, and benign summer lucitis.

26. The method according to claim 25, wherein the skin cancers are selected from the group formed by basocellular and spinocellular cancers or malignant melanoma.

27. (canceled)

28. The method according to claim 22, for promoting healing.

29. (canceled)

30. The method according to claim 23, wherein inflammatory diseases comprise psoriasis.

31. The method according to claim 22, for preventing and/or treating diseases selected from the group formed by gingivites and parodontites.

32. The method according to claim 22, for preventing and/or treating diseases selected from the group formed by vulvites and vaginites.

33. The method according to claim 22, for preventing and/or treating diseases selected from the group formed by arthritis and arthrosis.

34. (canceled)

35. (canceled)

36. The method according to claim 22, wherein the composition is administered via a topical or oral route.

37. The method according to claim 36, wherein the composition administered via a topical route comprises 0.001 to 50% by weight, of alpha-eleostearic acid, based on the total weight of said composition.

38. the method according to claim 37, wherein the composition administered via a topical route comprises 0.5 to 20% by weight of alpha leostearic acid, based on the total weight of said composition.

39. The method according to claim 36, wherein the composition administered via a topical route comprises 0.001 to 50% by weight, of alpha-eleostearic acid and 0.001 to 50% by weight, of catalpic acid, based on the total weight of said composition.

40. The method according to claim 39, wherein the composition administered via a topical route comprises 0.5 to 20% by weight of alpha-eleostearic acid and 0.5 to 20% by weight of catalpic acid, based on the total weight of said composition.

41. The method according to claim 36, wherein the composition administered via an oral route comprises 0.001 to 100% by weight, of alpha-eleostearic acid, based on the total weight of said composition.

42. The method according to claim 41, wherein the composition administered via an oral route comprises 1 to 50% by weight of alpha-eleostearic acid, based on the total weight of said composition.

43. The method according to claim 41, wherein the composition administered via an oral route comprises 0.001 to 100% by weight, of alpha-eleostearic acid, and 0.001 to 100% by weight, of catalpic acid, based on the total weight of said composition.

44. The method according to claim 43, wherein the composition administered via an oral route comprises 1 to 50% by weight of alpha-eleostearic acid, and 1 to 50% by weight of catalpic acid, based on the total weight of said composition.

45. The method according to claim 22, wherein the composition further comprises active ingredients selected from the group formed by anti-inflammatory agents.

46. The method according to claim 22, wherein the conjugated fatty acid source is a lipid extract of at least one plant selected from the group formed by plants of the Cucurbitaceae, Punicaceae, Bignoniaceae, Euphorbiaceae, Compositeae (Asteraceae), Balsaminaceae, Rosaceae, Chrysobalanaceae, Ricinocarpus and Chilopsis family.

47. The method according to claim 46, wherein the conjugated fatty acid source is a lipid extract of at least one plant selected from the group formed by green, white, pearl and wild Mormordicae, Catalpa, Aleurites, Euphorbia, Parinarium, Licania, Larinarium, Calendula, Punica, pomegranate tree, China wood. balsam, Trichosanthes, Centratus, and Jacaranda.

48. The method according to claim 47, wherein the source of alpha-eleostearic acid and catalpic acid, is a lipid extract of Momordica seeds.

49. The method according to claim 48, wherein the source of alpha-eleostearic acid and catalpic acid, is a lipid extract of Momordica charantia seeds.

50. A process for the preparation of liquid extract of Momordica consisting of extracting total lipids from Momordica seeds, dried and milled beforehand, by means of an oil solvent, and then evaporating said solvent.

51. A process for the preparation of liquid extract of Momordica consisting of extracting the lipids from Momordica seeds by cold mechanical pressing of the seeds.
